(19) United States
(12) Patent Application Publication
(10) Pub. No.: US 2014/0295054 A1
(43) Pub. Date: Oct. 2, 2014

(54) Molecular Film Containing Polymeric Mixture for Hydrophobic Implant Surfaces

(71) Applicant: ARIZONA BOARD OF REGENTS, Scottsdale, AZ (US)

(72) Inventors: Nicole Herbots, Tempe, AZ (US); Ashlee Murphy, Scottsdale, AZ (US); David Sell, Phoenix, AZ (US); Robert Culbertson, Tempe, AZ (US); Angelica S. Benitez, Phoenix, AZ (US); Tyler Kutz, Tempe, AZ (US); Ross Bennett-Kennett, Gilbert, AZ (US); Matthew Bade, Chandler, AZ (US); Brance P. Hudzeitz, Tempe, AZ (US)

(21) Appl. No.: 14/353,917

(22) PCT Filed: Oct. 26, 2012
(86) PCT No.: PCT/US2012/062196

§ 371 Date: Apr. 24, 2014

Related U.S. Application Data
(60) Provisional application No. 61/555,133, filed on Nov. 3, 2011.

(51) Int. Cl. A61L 27/26; A61L 27/50; A61L 27/34

(52) U.S. Cl. CPC A61L 27/26; A61L 27/34; A61L 27/50; A61L 2430/16; A61L 2400/18
USPC 427/2.25; 428/478.2; 206/570; 206/571; 220/529; 106/140.1; 514/13.7; 514/13.6; 424/94.64; 427/2.1; 427/2.24

(57) ABSTRACT

Compositions are disclosed containing a polymeric mixture diluted into an aqueous solution, which can be usefully applied to any surface mat is hydbcphoixc to act, for example, as an aotifoggiag coating with minimal optical distortion and excellent transparency. The compositions can also be used as lubricious agents on medical implants, shunts, and surgical supplies to minimize tissue trauma, to maximize bio-compatibility, and to increase healing by enhancing better irrigation and flow in adjacent tissue.

### RELATED APPLICATIONS

This application claims priority from U.S. Provisional Patent Application Ser. No. 61/555,133 filed Nov. 3, 2011, incorporated by reference herein in its entirety.

### BACKGROUND OF THE INVENTION

There are over fifteen million cataract surgeries performed every year worldwide, and the worldwide population diagnosed with Type II Diabetes at a high risk of retinopathy has reached sixty million, with six million in the United States alone. Patients will, unfortunately often suffer from retinal detachment after their initial surgery and have to undergo a secondary surgery. However, a secondary surgery performed after the implantation of artificial intra-ocular lenses (IOLs) due to retinal detachment often faces problems as a result of condensation on the surface of the lens. Although not all forms of IOLs are susceptible to this problem, the newer and higher quality models are (silicone and hydrophobic acrylic specifically). As a result, individuals with diabetes are unable to use the newer models due to problems with condensation.

A first complication is blood accumulation on IOLs during surgery to address trauma situations such as retinal detachments as well as diabetic capillary bleeding in the retina. Blood, blood proteins, and clots can interfere with surgical repair of the blood vessels and reattachment of the retina. This is notable because 1 in 6 Americans will suffer from type II diabetes in the course of his or her lifetime and 1 in 5 above age 45 will concurrently have IOLs implanted cataract surgery. This amounts to three million surgeries a year in the United States, making it the leading medical expense of Medicare. Cataracts are the leading cause of 60% of vision decline and blindness Similar statistics are derived from other countries such as Europe, Australia, Japan, and South Africa while developing countries where lifespan and illness have both rapidly increased in the last decades, such as India and China are also growing in cataract treatment and diabetes morbidity.

A second major problem in the medical field arises from condensation of the lenses. This is due to the evaporation of bodily fluids in bodily cavities during laparoscopic surgery and other microsurgical procedures. This latest advance in surgery, surgical practices and the increased usage of natural body orifices would minimize cutting, therefore minimizing recovery times, infections, hospital stays, surgical trauma and iatrogeny. At the Spring Convention of Laparoscopic Surgery in April 2011 in San Francisco, it was estimated that no less than 40% of the duration of surgery is spent removing condensation by wiping lenses. Presently, specialized sponges are used to wipe away condensation. However, their use requires frequent removal and reinsertion of surgical tool through the surgical port, which increases the risk of infection and additional trauma. Other surgeons warm up their tools prior to surgery, which lengthens the initial time it takes the surface to fog, but ultimately does not eliminate nor reduce the wiping time for surgeries, again increasing the risk of mine trauma and possible infection.

A third unresolved problem is protein and blood clotting and cell build up on medical implant devices and grafts. The accumulation of cell materials, clots and blood protein is a very significant issue in intervention such as blood vessel grafting. The goal is to avoid clot formation, which leads to thrombosis (stroke) and to avoid narrowing and obstruction of blood vessels, which can cause coronary heart disease, heart attacks, and cardiac infarction by preventing the accumulation of physiological deposits on medical implants and grafts. The present invention can significantly reduce post-surgical complications and specifically reduce the two-week “sudden death” rate observed in cardiac patients post-surgery.

To address these issues, and others, surface treatments that prevent the build-up of protein, blood, cell materials, as well as condensation a variety of surfaces that are used in medical implant is desirable.

### SUMMARY OF THE INVENTION

In a first aspect, the present invention provides compositions, comprising a mixture of (a) a first coagulant soluble plasma glycoprotein having a molecular weight of between about 50,000 Da and about 350,000 Da; (b) an optional second glycoprotein anti-coagulant that is an agonist of the first glycoprotein having a high negative charge density; and (c) a physiologically balanced aqueous solution containing a long chain repeating polymer having a molecular weight of between about 20,000 Da and 4,000,000 Da; wherein the volume ratios of (a) plus (b) compared to (c) is between about 1:3 and about 1:1500.

In a second aspect, the present invention provides a device, comprising or consisting of (a) an optional first layer comprising or consisting of a physiological aqueous solution containing a long chain repeating polymer having a molecular weight of between about 20,000 De and about 4,000,000 Da; (b) a second layer comprising or consisting of the composition of any embodiment of the first aspect of the invention in emulsion form; wherein when the first layer is present, the first layer and the second layer are in direct contact.

In a third aspect, the present invention provides kits comprising (a) a first container comprising or consisting of the soluble plasma glycoprotein component of any embodiment of the first aspect of the invention (b) a second container comprising or consisting of the against agent of the primary glycoprotein component of any embodiment of the first aspect of the invention; and (c) a third container comprising or consisting of the physiological aqueous solution component of any embodiment of the first aspect of the invention.

In a fourth aspect, the present invention provides methods for modifying a hydrophobic surface, comprising coating the hydrophobic surface with a device according to the second aspect of the invention, wherein either the first layer of the device, when present, is in direct contact with the hydrophobic surface, or the second layer of the device is in direct contact with the hydrophobic surface.

### BRIEF DESCRIPTION OF THE DRAWINGS

FIG. 1 is an illustration of an experimental setup used to simulate the condensation encountered in the human eye in surgery.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989. Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al 1990. Academic Press, San Diego, Calif.), Culture of Animal Cells: A Manual of Basic Technique, 2^{nd} Ed. (R. I. Freshney. 1987. Liss, Inc. New York, N.Y.). Gene Transfer and Expression Protocols, pp. 109-128, ed. E. J. Murray, The Humana Press Inc., Clifton, N.J.), and the Ambion 1998 Catalog (Ambion, Austin, Tex.).

All embodiments disclosed herein can be combined with other embodiments unless the context clearly dictates otherwise.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number, respectively. Additionally, the words “herein,” “above,” and “below” and words of similar import when used in this application, shall refer to this application as a whole and not to any particular portions of this application. As used herein, the singular forms “a”, “an” and “the” include plural referents unless the context clearly dictates otherwise. “And” as used herein is interchangeably used with “or” unless expressly stated otherwise.

As used herein, the term “about” means within 5% of the recited limitation.

The description of embodiments of the disclosure is not intended to be exhaustive or to limit the disclosure to the precise farm disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments.

In a first aspect, the present invention provides compositions, comprising a mixture of (a) a first coagulant soluble plasma glycoprotein having a molecular weight of between about 50,000 Da and about 350,000 Da; (b) an optional second glycoprotein anti-coagulant that is an agonist of the first glycoprotein, with a high negative charge density and (c) a physiological aqueous solution containing a long chain repeating polymer having a molecular weight of between about 20,000 Da and 4,000,000 Da; wherein the volume ratios of (a) plus (b) compared to (c) is between 1:3 and 1:1500.

The present invention includes a biocompatible polymeric mixture diluted in a physiologically balanced aqueous solution such as Balanced Salt Solution (BSS), Phosphate Buffered Solution (PBS), or using the molecular film as described in WO 2011/057275, filed Sep. 11, 2010, which is hereby incorporated by reference in its entirety. The polymeric mixture additionally includes one or more blood proteins such as fibrinogen that can be optionally mixed with other blood proteins such as heparin and albumin, and linear biopolymers such as vegetal hydrophilic polysaccharide like cellulose or an animal-source polypeptide like hyaluronic acid. This can occur in either polar or nonpolar combination pair or triple compounds.

The bio-compatible polymeric mixture of the present invention can be used, for example, either temporarily or permanently to control: 1) hydroaffinity, 2) blood protein adsorption, or 3) build-up and clot-formation on medical implant devices using Low Viscosity Polar Liquids (LVPLs). The invention provides the ability to, for example, control blood protein adsorption and hydroaffinity, as well as limiting build-up and clots on surfaces of medical implants.

In various exemplary embodiments the first coagulant soluble plasma glycoprotein has a molecular weight of between about 50,000 Da and about 300,000 Da; about 50,000 Da and about 250,000 Da; about 75,000 Da and about 350,000 Da; about 75,000 Da and about 300,000 Da about 75,000 De and about 250,000 Da; about 80,000 Da and about 350,000 Da; about 80,000 Da and about 300,000 Da; and about 80,000 Do and about 250,000 Da.

The long chain repeating polymer having a molecular weight of between about 20,000 Da and about 4,000,000 Da can be, for example, a viscoelastic polymer. As used herein, the “molecular weight” of a polymer refers to the weight-averaged molecular weight of the referenced polymer. As used herein, the term “viscoelastic” means that the component exhibits both viscous and elastic properties when undergoing deformation.

Polymers used can range from hyaluraonic acid (which can be extracted from animal tissue in various polymeric lengths, forms, purity and concentration) to plant-based cellulose in various polymeric lengths, forms, purity and concentration). Thus, for example non-limiting exemplary viscoelastic polymers that can be used in the compositions of the invention include, but are not limited to hyaluronic acid; hydroxypropylmethylcellulose (HPMC); hydroxyethylmethylcellulose and mixtures thereof or various combustions of shorter polymeric segments (oligomers) thereof.

In certain preferred embodiments, the physiological aqueous solution comprises between about 0.0003 wt % and about 10 wt % viscoelastic polymer. For example the physiological aqueous solution can contain between about 0.0003 wt % and about 5.0 wt %; or between about 0.0003 wt % and about 3.0 wt %; or between about 0.0003 wt % and about 2.0 wt %; or between about 0.0003 wt % and about 1.0 wt %; or between about 0.0005 wt % and about 10 wt %; or between about 0.0005 wt % and about 5.0 wt %; or between about 0.0005 wt % and about 3.0 wt %; or between about 0.0005 wt % and about 2.0 wt %; or between about 0.0005 wt % and about 1.0 wt %; between about 0.001 wt % and about 10 wt % or between about 0.001 wt % and about 5.0 wt % or between about 0.001 wt % and about 3.0 wt %; or between about 0.001 wt % and about 2.0 wt %; or between about 0.001 wt % and about 1.0 wt %; between about 0.01 wt % and about 10 wt %; or between about 0.01 wt % and about 5.0 wt % or between about 0.01 wt % and about 3.0 wt %; or between about 0.01 wt % and about 2.0 wt %; at between about 0.01 wt % and about 1.0 wt %; between about 0.1 wt % and about 10 wt %; or between about 0.1 wt % and about 5.0 wt %; or between about 0.1 wt % and about 3.0 wt %; or between about 0.1 wt % and about 2.0 wt %; or between about 0.1 wt % and about 1.0 wt % viscoelastic polymer.

The preferred polymer is solvated, which means that the polymer chains are surrounded by an essentially continuous molecular tube made of solvent molecules whose dipoles are aligned to form a solvation “cage” around the polymer chains to form “strands” (like strands of pearls where the polymer is the thread and the water molecules are the surrounding pearls). As used herein “solvated” means the solvent molecules associate with polymer chain by electrostatic dipole-dipole interactions between the solvent molecules and polymer components. Solvation of the polymer allows for the presence of ions in the gel, which can enhance conduction and electrostatic interaction along the polymer chains.

In another preferred embodiment, a commercial viscoelastic polymeric gel can be used for preparing the physiological aqueous solution. Non-limiting examples of commercial viscoelastic polymeric gels that can be adapted for use, for example, by the proper dilution in the present invention include any gel that has been FDA approved for eye surgery, including, but not limited to:
(1) OcuCoat® (Bausch & Lomb) comprises 2% 80 KDa Hydroxypropyl methylcellulose (20 mg/mL), sodium chloride (0.49%), potassium chloride (0.075%), calcium chloride (0.048%), magnesium chloride (0.03%), sodium acetate (0.39%), sodium citrate (0.17%), remainder water, the composition having a viscosity of 4000±1500 cst;
(2) Viscoat® (Alcon Laboratories) comprises a buffed solution of 3% 500,000 Da sodium hyaluronate (30 mg/mL) and 4% 22.5 KDa chondroitin sulfate, and has a viscosity of about 40000±20000 cps and a pH of 7.2±0.2;
(3) Healon® (Abbott Medical Optics) comprises 1% 4.0 MDa sodium hyaluronate (10 mg/mL) in a sodium/chloride/phosphate buffer, and has a viscosity of about 300,000 mPas and a pH of about 7.0-7.51;
(4) DuoVisc® (Alcon Laboratories) is a combination of Viscoat® and ProVisc® at varying ratios. Both Viscoat®, as listed above, and Provisc®, are sodium hyaluronate solutions a sodium/chloride/phosphate buffer;
(5) Amvisc® (Bausch & Lomb), comprises a solution of 1.2% 2.0 MDa sodium hyaluronate (16 mg/mL) in a physiological sodium chloride phosphate buffer solution (pH 6.8-7.6), and having a viscosity of about 132,000 cP at 25° C. and an osmolality of approximately 340 mOsmol;
(6) Amvisc® PLUS (Bausch & Lomb), comprises a solution of 1.6% 1.5 MDa sodium hyaluronate (16 mg/mL) in a physiological sodium chloride phosphate buffer solution (pH 6.8-7.6), and having a viscosity of about 132,000 cP at 25° C. and an osmolality of approximately 340 mOsmol;
(7) BioLon® (Bio-Technology General (Israel) Ltd.) comprises a solution of 1.0% 3 MDa sodium hyaluronate;
(8) Cellugel® (Alcon Labs) comprises a solution of 2.0% 300 KDa HPMC:
(9) CoEase® (Advanced Medical Optics, Inc.) comprises a solution of 1.2% 1 MDa sodium hyaluronate;
(10) EyeVisc™ (Biotech Visionare) comprises a solution of 2.0% HPMC:
(11) EyeVisc™ Pus (Biotech Visioncare) comprises a solution of 2.0% HPMC;
(12) EyeVisc™ SH (Biotech Visioncare) comprises a solution of 1.4% sodium hyaluronate;
(13) Healon® GV (Abbott Medical Optics) comprises a solution of 1.4% 5 MDa sodium hyaluronate;
(14) LensVisc™ MC (LensTec) comprises a solution of 2.0% 86 KDa HPMC;
(15) LensVisc™ HA (LensTec) comprises a solution of 2.0% 2.3 MDa sodium hyaluronate;
(16) Occu-Lon™ comprises a solution of 1.5% 2 MDa sodium hyaluronate;
(17) ShedGel® (Cytoslol Ophthamics, Inc) comprises a solution of 1.2% sodium hyaluronate;
(18) STAARVisc® II (STAAR Surgical Company) comprises a solution of 1.2% sodium hyaluronate;
(19) UniVisc™ (CIBA Vision) comprises a solution of 1.0% 3 MDa sodium hyaluronate; and
(20) Vitrax (Abbott Medical Optics) comprises a solution of 3.0% 500 KDa sodium hyaluronate.

Sea, for example, Rice, D. J et al., C. J., Ophthalmologic Drug Guide, Springer New York, 2007, pages 90-91, which is hereby incorporated by reference.

In certain preferred embodiments, the physiological aqueous solution comprises Viscoat®, Amvisc®, or Duovisc®. Far example, a preferred embodiment of a viscoelastic polymeric gel is Healon®, FDA approved for eye surgery, which comprises fully solvated hyaluronic acid (10 mg/mL). In another preferred embodiment, the physiological aqueous solution comprises HPMC (e.g., OcuCoat®).

When a commercial viscoelastic polymeric gel is used for preparing a physiological aqueous solution, it may diluted with an ionically conductive aqueous solution to provide a suitable polymer concentration as noted above. The “ionically conductive aqueous solution” can be any suitable fluid comprising an aqueous electrolyte, such as any saline solution. In one preferred embodiment, the electrolyte in the ionically conductive aqueous comprise at least 0.03% (one ion to about 3000 water molecules) of the solution. In mother preferred embodiment, the electrolyte in the ionically conductive aqueous comprise between at least 0.03% to about 0.5% of the solution; in further preferred embodiments, between at least 0.05% and about 0.4%; between at least 0.03% and about 0.3%; and at least 0.03% and about 0.2% of the solution.

The volume ratio of the commercial viscoelastic polymeric gel to the ionically conductive aqueous solution is between about 1:3 and about 1:1500 depending on the molecular weight of the polymer in the solvated gel. For lower molecular weight polymers, such as HPMC, a ratio of about 1:3 or about 1:5 works well. For higher molecular weight polymers, such as hyaluronic acid, it is best to emulsify the solvated polymeric gel at a higher dilution ratio. In various preferred embodiments the ratio is between about 1:3 and about 1:1000; about 1:3 to about 1:500; about 1:3 to about 1:250; about 1:3 to about 1:100; about 1:3 to about 1:50; about 1:3 to about 1:20; about 1:3 to about 1:10; about 1:3 to about 1:5; about 1:5 and about 1:1500; about 1:5 and about 1:1000; about 1:5 to about 1:500; about 1:5 to about 1:250; about 1:5 to about 1:100; about 1:5 to about 1:50; about 1:5 to about 1:20; about 1:5 to about 1:10; about 1:10 and about 1:1500; about 1:10 and about 1:1000; about 1:10 to about 1:500; about 1:10 to about 1:250; about 1:10 to about 1:100; about 1:10 to about 1:50; and about 1:10 to about 1:20. In one preferred embodiment for containing efficacy and uniformity of distribution of the fully solvated polymeric strands, the range is between about 1:3 to about 1:100. In a further preferred embodiment, the range is between about 1:10 and about 1:20.

In a further preferred embodiment, hydroxypropylmethylcellulose (HPMC) of molecular weight 86,000 Da is used at 2% by weight in saline, resulting in solvated viscoelastic polymeric gel having a viscosity of about 4000 cP.

In a further preferred embodiment a 2.5% HPMC (86,000 Da) gel by weight in saline exhibits a viscosity of 15,000 cP. Although a higher viscosity than preferred for fanning the physiological aqueous solution, higher concentration physiological aqueous solutions have value, for example, by extending the time to condensation, when used in the devices and methods of the invention (see below). In a further embodiment, 120,000 Da HPMC, a vitreous substitute, can be hydrated in a physiological aqueous solution to a concentration of 2% by weight in saline, prior to mixture with an ionically conductive aqueous solution to yield a matching viscosity of about 15,000 cP.

In one non-limiting exemplary embodiment, the physiological aqueous solution is as described for preparing the molecular film of WO 2011/057275, mixed with Fibrinogen (a glycoprotein with a molecular weight of 340,000 Da) and Heparin. Embodiments of the physiological aqueous solution described in WO 2011/057275 include, for example, a physiological aqueous solution comprising or consisting of a solution with the following characteristics (Table 1):

TABLE 1

| Component | |
|---|---|
| Na⁺ | 144 to 164 meq/L |
| K⁺ | 2 to 10.1 meq/L |
| Ca²⁺ | 1 to 3.3 meq/L |
| Mg²⁺ | 0.0 to 1.5 meq/L |
| Cl⁻ | 128 to 177 meq/L |
| HCO₃⁻ | 0.0 to 20 meq/L |
| Phosphate | 0.0 to 3.0 meq/L |
| Lactate | 0.0 to 8.0 meq/L |
| Glucose | 0.0 to 5.0 meq/L |
| Ascorbate | 0.0 to 2.0 meq/L |
| Glutathione | 0.0 to 0.3 meq/L |
| Citrate | 0.0 to 6.0 meq/L |
| Acetate | 0.0 to 3.0 meq/L |
| pH | 7.1 to 7.7 |
| Osmolality | 250 to 350 mOsmoles/L |

Thus, in one preferred embodiment, the physiological aqueous solution comprises or consists of Na⁺, K⁺, Ca²⁺, Mg²⁺, and Cl⁻ within the range noted in Table 1, and has a pH and osmolality within the ranges noted in Table 1. The other components may be optionally added either individually or in any combination to prepare the final ionically conductive aqueous solution.

In another preferred embodiment, a physiological aqueous solution approximating (+/−10% for each component) the composition of human vitreous humor, approximating the composition of human aqueous humor, or any saline solution used in eye surgery or therapy, can be used (Table 2). In further preferred embodiments, the physiological aqueous solution is a solution comprising or consisting essentially of or consisting of components +/−9%, 8%, 7%, 6%, 5%, 4%, 3%, 2%, 1%, or identical to those listed in a solution listed in Table 2.

TABLE 2

| | | | | BSS | |
|---|---|---|---|---|---|
| | | | Hartman's | PLUS ® | BSS ® |
| | Human | Human | Lactated | Intraocular | Intraocular |
| Ingredient | Aqueous | Vitreous | Ringer's | Irrigating | Irrigating |
| in meq/L | Humor | Humor | Solution | Solution | Solution |
| Sodium | 162.9 | 144 | 102 | 160.0 | 155.7 |
| Potassium | 2.2-3.9 | 5.5 | 4 | 5.0 | 10.1 |
| Calcium | 1.8 | 1.6 | 3 | 1.0 | 3.3 |
| Magnesium | 1.1 | 13 | — | 1.0 | 1.5 |
| Chloride | 131.6 | 177.0 | — | 130.0 | 128.9 |
| Bicarbonate | 20.15 | 15.0 | — | 25.0 | — |
| Phosphate | 0.62 | 0.4 | — | 3.0 | — |
| Lactate | 2.5 | 7.8 | 28 | — | — |
| Glucose | 2.7-3.7 | 3.4 | — | 5.0 | — |
| Ascorbate | 1.06 | 2.0 | — | — | — |
| Glutathione | 0.0019 | — | — | 0.3 | — |
| Citrate | — | — | — | — | 5.8 |
| Acetate | — | — | — | — | 28.6 |
| pH | 7.38 | — | 6.0-7.2 | 7.4 | 7.6 |
| Osmolatity | 304 | — | 277 | 305 | 298 |
| (mOsm) | | | | | |

Various further preferred embodiments for the physiological aqueous solution are those that approximate the compostions of body fluid compartments, for example, those with the ions and protein additives as listed in Table 3 below. The extracellular fluid compartment (ECF) is composed of the blood/plasma compartment and the interstitial fluid compartment.

TABLE 3

| Fluid | | |
|---|---|---|
| Compartment | Component | Composition elements |
| Extracellular | Blood/plasma | Na⁺, K⁺, Ca²⁺, Mg²⁺, HCO₃⁻, |
| | | Cl⁻, HPO₄²⁻, SO₄²⁻, organic |
| | | acids, protein, non-electrolytes |
| | Interstitial | Na⁺, K⁺, Ca²⁺, Mg²⁺, HCO₃⁻, |
| | | Cl⁻, HPO₄²⁻, SO₄²⁻, organic |
| | | acids, protein, non-electrolytes |
| Intracellular | Cells | K⁺, Na⁺, Mg²⁺, HCO₃⁻, Cl⁻, |
| | | PO₄³⁻, SO₄²⁻, |

In one embodiment, the physiological aqueous solution is mixed with 3 mg/mL fibrinogen. Heparin can be provided form a heparin sodium injection vial with 20,000 USP units/mL, diluted with a balanced salt solution with a dilution ratio between about 1:1 and 1000:1 (BS:Sheparin).

In another preferred embodiment, the primary glycoprotein can comprise thrombin, hemoglobin, or albumin, while the secondary glycoprotein can comprise any blood thinner. In a third preferred embodiment, the secondary glycoprotein can comprise of warfarin or thrombin.

In yet another preferred embodiment, the aqueous solution can comprise a commercially available ionically conductive aqueous solution, such as, but not limited to, balanced saline solution (BSS) from Akom (IL) and/or BSS intraocular irrigating solution (Alcon Laboratories, Inc., Fort Worth, Tex.). For applications in human device implants and medical implants, normal saline is a preferred electrolyte of choice. Far application to the most delicate tissues and to minimize inflammation and promote tissue healing after implantation balanced saline solution is a preferred ionically conductive aqueous solution.

In a second aspect, the present invention provides a device, comprising or consisting of (a) an optional first layer comprising or consisting of a physiological aqueous solution containing a long chain repeating polymer having a molecular weight of between about 20,000 Da and about 4,000,000 Da; and (b) a second layer comprising or consisting of the composition of any embodiment or combination of embodiments of the first aspect of the invention in emulsion farm; wherein when the first layer is present, the first layer and the second layer are in direct contact.

The devices according to this second aspect of the invention can be used, for example, to modify hydrophobic surfaces, such as medical devices, as described in more detail below. All embodiments and combinations of embodiments of the first aspect of the invention can be used in this aspect. Similarly, all embodiments or combinations of embodiments of the first layer, when present, can be used in the devices of this second aspect of the invention.

In a third aspect, the present invention provides kits comprising (a) a first container comprising or consisting of the soluble plasma glycoprotein component of any embodiment or combination of embodiments of the first aspect of the invention (b) a second container comprising or consisting of the agonist of the primary glycoprotein component of any embodiment or combination of embodiments of the first aspect of the invention and (c) a third container comprising or consisting of the physiological aqueous solution component of any embodiment or combination of embodiments of the first aspect of the invention. The kits can be used for any suitable purpose, such as for a kit user to prepare the compositions or devices of the present invention, and to carry out the methods of the present invention. The containers may be of any type suitable for a given purpose. In one embodiment, each container is a completely separate container. In another embodiment, at least one container comprises a partition (which may be removable) to segregate two of the kit components until a user is ready to prepare the compositions. In another embodiment, 1, 2, or all 3 of the containers is a syringe, and the kit further comprises one or more needles (i.e.: 1, 2, or 3 needles) that mate with the syringes for delivery of the components as desired.

The kits of the invention may further comprise any other components as suitable for a given use, such as sterilization means, including but not limited to ultra-violet (UV) light sources and/or heating lamps that can be used, for example, to sterilize the compositions and devices prior to use (such as for implantable medical devices), by subjecting the devices to UV light at wavelengths and under suitable conditions to kill bacteria and fungi, and to remove organic contaminants (i.e.: protein, nucleic acid, etc.).

In a fourth aspect, the present invention provides methods for coating a hydrophobic surface, comprising coating the hydrophobic surface with a device according to the second aspect of the invention, wherein either the first layer of the device, when present, is in direct contact with the hydrophobic surface or the second layer of the device is in direct contact with the hydrophobic surface. The hydrophobic surface may be any an which the device of the invention can be usefully applied as a coating, including but not limited to silicone, hydrophobic acrylic, any form of silicon dioxide, quartz or silicon substrates used for medical device implants and surgical supplies, shunts, and tubing and eyewear, such as sports visors, eye glasses, and goggles, having, for example high impact resistance coating such as a silicate over an underlying polycarbonate substrate.

The methods of the present invention can be used, for example, to control blood protein adsorption, build-up, and clot formation on implantable medical devices and grafts, as well as to control hydroaffinity and thereby limit fluid condemnation that can result in wetting (transparent) or fogging (non-transparent) on other types of hydrophobic surfaces.

In various non-limiting embodiments, the methods are used to coat a medical implant surface or a borosilicate lens used for visualization during laparoscopic surgery, or to view the surface of an Intraocular Lenticular implant (IOL), which is used in cataract surgery or in any situation in which bodily fluids such as blood, blood serum, blood proteins and clots are either accumulated or already present. The present invention limits/prevents fluid condensation with fogging (i.e.: discrete droplet nucleation), which leads to an opaque film that interferes with visualization, optical signals, and optical sensing. The methods of the invention also prevent/limit trapping of infectious agents and spurious cell, protein, or biological material debris on medical implants and visualizing lenses, such as those used in laparoscopic surgery.

The coating can be applied as either removable or permanent surface bio-compatible adsorbate coatings. In one exemplary embodiment, if protein adsorption, subsequent cell adhesion and clot formation has to be limited/prevented only during surgery on laparoscopic visualization lenses, IOLs or medical implants, a temporary coating can be applied. In another non-limiting embodiment, a permanent coating can be applied if for example, the medical implant has to remain free of tissue adhesion, such as a chemotherapy pump implanted for the duration of a cancer treatment or a dialysis part used until the patient is able to obtain a permanent kidney transplant. In cases where protein and cell build up is advantageous for medical implants, the coatings can be removed a absorbed via an enzymatic agent applied to the surface, or sonication can be used to remove the coating from medical tools.

The coating may be applied to a surface as a mixture of the three components simultaneously. However, each component can also be applied separately, in turn and in any order. In some embodiments, only one of the first glycoproteins and a physiologically balanced aqueous solution are used.

The first coagulant soluble plasma glycoprotein, the optional second glycoprotein anti-coagulant that is an agonist of the first glycoprotein, and the physiological aqueous solution for use in the methods of the invention can be any embodiment or combination of embodiments of these components disclosed in the first and second aspects of the invention.

In one preferred embodiment, the emulsion comprises or consists of three components as shown in Table 4. The first component is a soluble plasma glycoprotein that is a coagulant and hydrophilic having a weight range of 50,000 to 350,000 Da. An example of the first component is fibrinogen. In an exemplary embodiment, fibrinogen with 3 mg/mL is used. In other embodiments, the fibrinogen with a dilution between approximately 0.5 mg/mL and 18 mg/mL can be used. In addition, the following is a list of alternatives with their respective approximated concentrations normally found in blood: 1) thrombin—0.15 to 0.20 mg/mL, 2) hemoglobin—120 to 180 mg/mL, and 3) albumin—24 to 54 mg/mL. For these components, a concentration approximately of the same order as normal physiological levels found in the human body (e.g. in blood) may be used.

The second component contains an agonist of the first and other agents, and is characterized as an anti-coagulant with a high negative charge density. An example of such a material is heparin, which may be diluted with BSS. In an exemplary embodiment, heparin can be provided from a heparin sodium injection vial with 20,000 USP units/mL, and is diluted with a balanced salt solution (BSS) with a dilution ratio between approximately 1:1 and 1000:1 (BSS:heparin) by volume. Alternatives to the second component include, but are not limited to, any blood thinner such as, but not limited to ibuprofen, warfarin and thrombin. The last component is a physiologically balanced aqueous solution including any embodiment or combination of embodiments disclosed above. Alternatives to the third component include, but are not limited to, BSS and PBS.

TABLE 4

| | | | Dilution | Substitutes |
|---|---|---|---|---|
| | | | Range for | and Alter- |
| | | | the Example | natives to |
| | Key | | Provided | the Examples |
| Component | Properties | Example | (mg/mL) | Provided |
| Primary | Coagulant | Fibrinogen | 0.5-18 mg/mL | Hemoglobin |
| Soluble | Hydrophilic | | | Albumin |
| Plasma | 50,000 to | | | Thrombin |
| Glyco- | 350,000 Da | | | |
| protein | | | | |
| Secondary | Anti- | Heparin | 20,000 USP | Blood |
| Glyco- | coagulant | | units/mL, | thinners |
| protein | Agonist | | diluted in | Warfarin |
| | agent of | | Balanced Salt | Thrombin |
| | Primary | | Solution | |
| | Glycoprotein | | (BSS:Heparin) | |
| | High | | at ranges | |
| | Negative | | between 1:1 | |
| | Charge | | and 1000:1 | |
| | Density | | | |
| Physio- | Contains a | Molecular | N/A | Balanced |
| logical | long chain | film de- | | Salt Solution |
| Balanced | repeating | scribed in | | Phosphate |
| Aqueous | polymer | described | | Buffered |
| Solotion | 20,000 to | in WO | | Solution |
| | 4,000,000 | 2011/ | | |
| | Da | 057275 | | |

In a further embodiment, the methods comprise sterilization of the compositions or devices for a time and under conditions suitable to kill bacteria and fungi, and/or to remove organic contaminants (i.e.: protein, nucleic acid, etc.). Any suitable sterilization technique can be used. In one embodiment. UV sterilization is used, under any suitable conditions to kill fungi/bacteria and to destroy organic contaminants without damaging the polymeric mesh. In one exemplary, non-limiting embodiment, UV treatment is carried out at two different wavelengths, such as between 254 nm to 254.7 nm to fill fungi/bacteria and 185 nm to destroy organic contaminant, using a suitable UV light source (including but not limited to an He—Ne bulb). In another embodiment, UV treatment can be carried out only at the 254 nm to 254.7 nm wavelengths. Those of skill in the art will recognize that other UV wavelengths may also be used UV sterilization treatment can be carried out for any suitable time period, such as between 2 seconds to 60 seconds, 10 seconds to 60 seconds, and 2 seconds to 10 seconds. It will be apparent to those of skill in the art that the duration of the UV treatment will depend on a variety of factors, including molecular weight of polymers in the compositions or devices.

### Example 1

### Procedure

Using silicone HD500 and hydrophobic acrylic intra-ocular lenses three different compounds as described in Table 1 were applied in layers and mixtures in different dilutions (with 18 mg/mL as highest concentration). The dilution level affected only hydroxypropyl methylcellulose (HPMC); it stopped condensation an the intra-ocular lenses around a 5:1 dilution with Balanced Salt Solution (BSS). Heparin was diluted with Balanced Salt Solution to 512:1 and it did not change in its behavior. Fibrinogen also showed no behavioral change, although it should be noted that in the lab it was diluted only a small amount, 8:1 with BSS.

Hydroxypropyl methylcelluose (HPMC) is an inert viscoelastic polymer, which forms a colloid when mixed with water HPMC is non-polar which allows it to interact with the hydrophobic surfaces of silicone or hydrophobic acrylic lenses. However, water molecules also uniformly coat its surface allowing it to form a hydrophilic layer over the surface on which it was applied, preventing any optically interfering condensation from forming. Currently, this solution is at the top of the list for anti-condensation applications to be used during retinal surgery due to its inert behavior in biological systems and ease of application. This applicability, however, requires that there are no common elements inside of an eye during surgery that can hinder its effectiveness. The previous tests with blood proteins suggest that there may not be a problem at all with the blood proteins. The nature of the problem may arise because the doctor can use Ocucoat® (2% hydroxypropyl methylcellulose in a buffered solution) and manually dilute it in balanced saline solution during surgery. This process takes time and introduces more human error, and it is these mistakes in the process that are suspected to be the came of failed usage.

The first blood protein tested on the intra-ocular lenses was heparin, which is a particularly potent anti-coagulant and has the highest negative charge density of any known biological molecule. Extensive tests on heparin's interaction on the surface of the hydrophobic lenses implied that the molecule's interaction with the surface is nearly identical to that of water. The proteins did not absorb onto the surface (as shown in ion beam analysis), and effectively did nothing to prevent fogging from occurring.

Fibrinogen was applied to assess its properties and interactions. Fibrinogen does not have an extreme charge density on either end of the spectrum. It has both polar and non-polar components allowing it to interact with both hydrophobic and hydrophilic surfaces. When fibrinogen was applied on a hydrophobic acrylic lens, an inhibition of condensation occurred. However, unlike HPMC, this inhibition effect could not be removed. It was washed with 18 mega ohm deionized water as well as SC1 and the layer still remained on the lens surface during the second round of testing. It was later discovered that fibrinogen absorbed into the surface and papers on the subject implied that the process was irreversible. Given that the absorption process likely results in the denaturing of the protein, it is believed that this could be used in the manufacturing process of the lenses to permanently prevent condensation from occurring during surgery.

### Materials and Methods

Condensation tests were used as a direct measurement of a specific coating on the surface of the lenses.

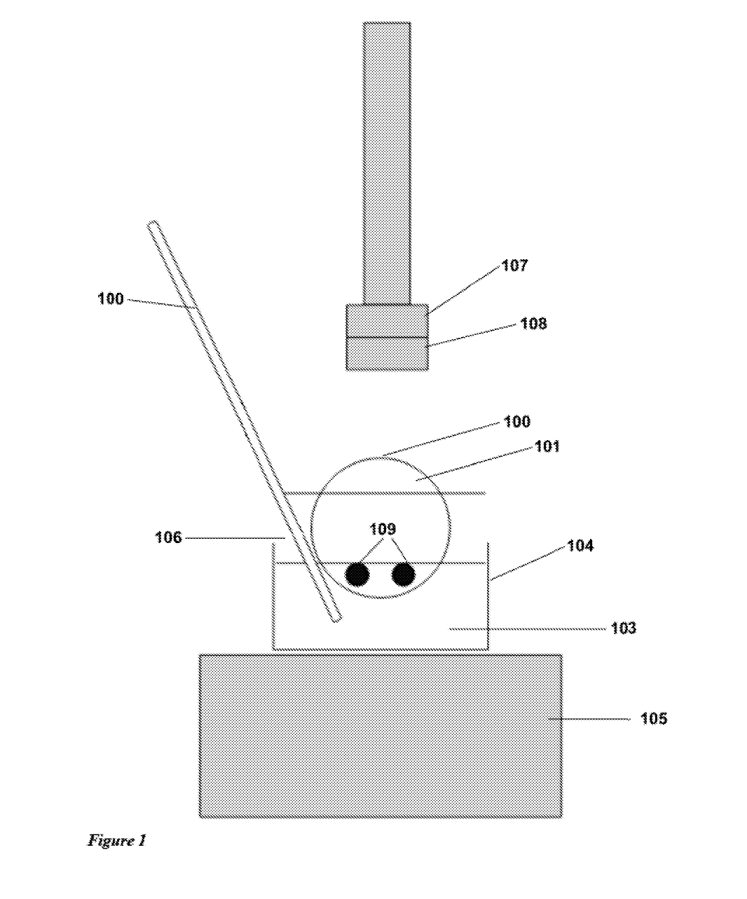

Coated lenses (100) were mounted on an artificial eyeball (101) in a small condensation chamber (102) as illustrated in FIG. 1. Water vapor is produced by heating DI water or Balanced Salt Solution (BSS), (103) in a petri dish (104) over a hot plate (105) to a temperature of 38° C., which was monitored by a mounted thermometer (106). The vapor reaches the surface of the lenses to simulate the condensation encountered in the human eye in surgery. Small holes (109) are present in the condensation chamber which is partially immersed in the 38° C. water heated in the petri dish. These holes are present in the condensation chamber supporting the lens to let water vapor from the heated water rise inside the condensation chamber and thus simulate condensation on the side of the lens facing down towards the inside of the eye in the same configuration and geometry encountered during surgery. Holes are also present in the top for the lens and a hole an the side slightly under the elevation of the top for needle insertion (not shown). The surface of the lenses was monitored by a camera (107) and optional microscope (108) mounted above the observed surface.

The lens is placed on the top of the artificial eye and a needle is inserted through the hole on the side containing the substance to be tested and deposited on the lower surface of the lens. A separate needle is used to reduce the thickness of the layer on the lens's surface.

After the layer reduction is done, the time it takes for condensation to inhibit vision in the center of the lens is approximated with a stopwach. If a given substance is able to prevent condensation for 20 mines, or longer depending on whether that substance has been tested before, the test is cut off and it is noted that the coating successfully prevented vision inhibiting condensation from forming. The time of 20 minutes is chosen because it is double the time the retinal surgery in question takes, which is approximately 10 minutes.

HPMC has a 100% success rate in the lab, however, after the first four clinical trials resulted in two failures. Possible causes other than preparation error were investigated, the prominent one being inhibition by blood proteins. Furthermore, it was also noted that surgeries done an lens types susceptible to the condensation problem did not always inhibit vision during surgery, so tests were done on the direct impact of these blood proteins onto the lens as well.

The two blood proteins that were tested were heparin and fibrinogen. The hypothesis was that neither molecule should inhibit the effectiveness of HPMC or have any effect on the surface condensation properties given that both molecules are polar enough to be soluble in water. Therefore, the expected result was that the blood proteins would bead an the surface of the lenses, and de-wet during layer thinning rather than form a film, allowing condensation to occur as if the lens were dry.

The emulsion may be applied to a surface in a single step as a mixture of the three components. However, it may also be applied separately using the emulsion components individually. In an exemplary embodiment, fibrinogen may be applied to a surface to foam a complete, conformal coating to a surface. The surface is completely saturated by the fibrinogen and excess fibrinogen is removed, to leave a layer that is 10¹⁵ atoms/cm². Heparin may then be applied to form a conformal layer followed by the physiologically balanced solution.

When fibrinogen was applied to a surface, the lower dilutions (up to 6 mg/mL) showed no indication of opaqueness. On the other hand, the lenses seemed to develop an opaqueness issue when tested with a fibrinogen concentration of 18 mg/mL.

### Results and Discussion

When the heparin solution was applied to the lens, it would not spread an the surface, and would de-wet if extracted. In essence, no difference in behavior was observed between heparin and water on the surface of the lens, and no negative effects were observed when used in conjunction with HPMC. Furthermore, ion beam analysis on a Si(100) wafer which had been exposed to heparin and then allowed to de-wet suggested that no residue had absorbed onto the surface.

In contrast, fibrinogen drastically deviated from expected results. Although fibrinogen is soluble in water, a wetting effect on the surface of the lens was still observed, successfully preventing condensation on the lens without fail even with the low dilution of 1 mg/mL.

These effects could be due to the structures of heparin and fibrinogen. Heparin is a highly sulfated glycosaminoglycan, which is often used as an anticoagulant. Heparin, which has a molecular weight of about 3,000 Da, consists of a variably sulfated repeating disaccharide unit. Of any known biological molecule, heparin has the highest negative charge density. Heparin was chosen as an experimental medium due to its use as an anticoagulant on the surface of various experimental and medical devices in the hopes that it would prevent condensation on the Intra-Ocular Lenses also. The hexamer fibrinogen is a soluble plasma glycoprotein, which is synthesized by the liver. Fibrinogen's molecular weight is about 340,000 Da and its major function is to be the precursor to fibrin, which it is converted into by thrombin during blood coagulation.

Heparin was analyzed using Rutherford Backscattering Spectrometry (RBS) which is an analytical technique used to determine the structure and composition of materials by measuring the backscattering of a high-energy ion beam Heparin was also analyzed using Proton-Induced X ray emission (PIXE), which is used to determine the elemental make up of a sample in a non-destructive analysis PIXE exposes the sample to an ion beam and the atomic interactions that occur give off wavelengths in the x-ray part of the electromagnetic spectrum. Fibrinogen's composition does not have any unique heavy elements. It contains oxygen carbon and hydrogen like the Intra-Ocular lenses, therefore, it is not possible for Rutherford Back Scattering to detect. In the case of heparin RBS could not resolve the sulphur and the layer was applied too thickly. The observations of fibrinogen provided a possible explanation as to why the surgeons do not always report condensation on hydrophobic IOLs during surgery.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby. Each referenced document herein is incorporated by reference in its entirety for all purposes.

Changes can be made in the composition, operation and arrangement of the method of the present invention described herein without departing from the concept and scope of the invention as defined in the following claims.

We claim:

1. A composition, comprising a mixture of:
(a) a first coagulant soluble plasma glycoprotein having a molecular weight of between about 50,000 Da and about 350,000 Da;
(b) an optional second glycoprotein anti-coagulant that is an agonist of the first glycoprotein having a high negative charge density; and
(c) a physiologically balanced aqueous solution containing a long chain repeating polymer having a molecular weight of between about 20,000 Da and 4,000,000 Da; wherein the volume ratios of (a) plus (b) compared to (c) is between about 1:3 and about 1:1500.

2. The composition of claim 1, wherein the composition is an emulsion.

3. The composition of claim 1 wherein the first glycoprotein is fibrinogen, thrombin, hemoglobin, albumin, or a mixture thereof.

4. The composition of claim 1 wherein the second glycoprotein is a blood thinner.

5. The composition of claim 1 wherein the second glycoprotein is heparin, warfarin, or thrombin.

6. The composition of claim 1 comprising 20,000 USP units/mL of the second glycoprotein.

7. The composition of claim 1 wherein the second glycoprotein is diluted with a balanced salt solution with a dilution ratio between 1:1 and 1000:1 (BSS:Secondary glycoprotein).

8. The composition of claim 1 wherein the aqueous solution comprises a solution selected from the group consisting of:
(a) a balanced salt solution,
(b) a phosphate buffered solution,
(c) a solution comprising Na⁺, K⁺, Ca²⁺, Mg²⁺, and Cl⁻ within the range noted in Table 1, with a pH and osmolality within the ranges noted in Table 1; and
(d) a solution comprising components listed in Table 2, wherein each component is present in the solution within 10% of the amount listed in Table 2.

9. The composition of claim 1, wherein the volume ratios of (a) plus (b) compared to (c) is between about 1:3 and about 1:100.

10. The composition of claim 1, wherein the volume ratios of (a) plus (b) compared to (c) is between about 1:10 and about 1:20.

11. A device, consisting of
(a) an optional first layer comprising a physiological aqueous solution containing a long chain repeating polymer having a molecular weight of between about 20,000 Da and about 4,000,000 Da;
(b) a second layer comprising the composition of claim 1;
wherein when the first layer is present, the first layer and the second layer are in direct contact.

12. A kit comprising
(a) a first container comprising a first coagulant soluble plasma glycoprotein having a molecular weight of between about 50,000 Da and about 350,000 Da;
(b) a second container comprising a second glycoprotein anti-coagulant that is an agonist of the first glycoprotein having a high negative charge density; and
(c) a third container comprising a physiologically balanced aqueous solution containing a long chain repeating polymer having a molecular weight of between about 20,000 Da and 4,000,000 Da.

13. The kit of claim 12, wherein the first and second containers are separate compartments in a single container, separated by a removable partition.

14. The kit of claim 12, wherein the first and second containers are syringes.

15. A method for modifying a hydrophobic surface, comprising coating the hydrophobic surface with a device according to claim 11, wherein either the first layer of the device, when present, is in direct contact with the hydrophobic surface, or the second layer of the device is in direct contact with the hydrophobic surface.

16. A method for processing an intra-ocular lens (IOL), comprising
(a) optionally forming a first layer on a surface of the IOL by contacting the surface with an ionically conductive aqueous solution; and
(b) contacting the first layer, when present, or the surface, with a second layer comprising the composition according to claim 1.

17. A method for modifying a hydrophobic surface, comprising coating the hydrophobic surface with a composition according to claim 1, wherein only one of the first two parts: either (a) or (b), are used.

18. A method for modifying a hydrophobic surface, comprising coating the hydrophobic surface with a composition according to claim 1, wherein the components are applied simultaneously as a mixture of (a), (b), and (c), or they are applied separately in any order.

19. The method of claim 15, wherein the hydrophobic surface is selected from the group consisting of silicone, hydrophobic acrylic, silicon dioxide, quartz, and silicon surfaces.

20. The method of claim 15, wherein the hydrophobic surface is part of a device selected from the group consisting of medical device implants, surgical supplies, shunts, tubing, and eyewear.
